(19) United States
(12) Patent Application Publication
(10) Pub. No.: US 2021/0205340 A1
(43) Pub. Date: Jul. 8, 2021

(54) COMPOSITIONS COMPRISING SIALYLATED OLIGOSACCHARIDES FOR USE IN INFANTS OR YOUNG CHILDREN TO PREVENT LATER IN LIFE OBESITY OR RELATED COMORBIDITIES AND PROMOTE A HEALTHY GROWTH

(71) Applicant: NESTEC S.A., Vevey (CH)

(72) Inventors: Jose Manuel Ramos Nieves, Lausanne (CH); Aristea Binia, Fribourg (CH); Norbert Sprenger, Savigny (CH); Sami Damak, Lausanne (CH); Carole De Senarclens-Bezencon, Riex (CH)

(21) Appl. No.: 16/071,323

(22) PCT Filed: Jan. 26, 2017
(86) PCT No.: PCT/EP2017/051581

§ 371 Date: Jul. 19, 2018

(30) Foreign Application Priority Data
Jan. 26, 2016 (EP) 16152744.5

(51) Int. Cl. A61K 31/702; A23L 33/00; A23L 29/30; A23L 33/155; A23L 33/29; A23L 33/115; A23L 33/19; A23L 33/135

(52) U.S. Cl. CPC A61K 31/702; A23L 33/30; A23L 33/40; A23L 29/30; A23L 33/135; A23L 33/29; A23L 33/115; A23L 33/19; A23L 33/155

(57) ABSTRACT

The present invention relates to a nutritional composition comprising at least one sialylated oligosaccharide, for use in reducing and/or avoiding excessive fat mass accumulation, in preventing the associated later in life health disorders like later in life obesity and related comorbidities, in reducing and/or controlling food intake and/or in promoting a healthy growth in an infant or a young child.

### FIELD OF THE INVENTION

This invention relates to nutritional compositions comprising specific human milk oligosaccharides for use in avoiding excessive fat mass accumulation, in controlling food intake and/or in promoting a healthy growth in an infant or a young child. They especially allow to prevent obesity later in life in infants or young children.

### BACKGROUND OF THE INVENTION

Overweight and obesity are defined as abnormal or excessive fat accumulation that may impair health. Body mass index (BMI) is a simple index of weight-for-height that is commonly used to classify overweight and obesity. It is defined as a person's weight in kilograms divided by the square of his height in meters (kg/m2). The WHO definition is: a BMI greater than or equal to 25 is overweight; a BMI greater than or equal to 30 is obesity.

The prevalence of obesity and overweight in adults, children and adolescents has increased rapidly over the past 30 years globally and continues to rise. Worldwide obesity has more than doubled since 1980, as reported by the WHO. It has become a global health concern since it is associated with a reduced life time, an altered life quality and it is responsible of further health conditions. Overweight and obesity are linked to more deaths worldwide than underweight. Childhood obesity is indeed associated with a higher chance of obesity, premature death and disability in adulthood. But in addition to increased future risks, obese children experience breathing difficulties, increased risk of fractures, hypertension, early markers of cardiovascular disease, insulin resistance and psychological effects. Raised BMI is a major risk factor for noncommunicable diseases such as cardiovascular diseases (mainly heart disease and stroke), which were the leading cause of death in 2012; diabetes; musculoskeletal disorders (especially osteoarthritis—a highly disabling degenerative disease of the joints); even some cancers (endometrial, breast, and colon).

Evidence suggests that infancy may be a critical period in the development and programming of obesity or future related comorbidities (including the metabolic disorders). Mother's milk is recommended for all infants for various reasons. Breastfeeding has especially been reported to be beneficial for prevention against obesity in comparison to formula feeding (Owen et al, Effect of Infant Feeding on the Risk of Obesity Across the Life Course: A Quantitative Review of Published Evidence, 2005). It has been widely reported that breast fed infants do have a different growth pattern than infants fed with infant formula. Indeed, breast fed infants have a lower weight gain and a lower body fat mass within the first year of life as compared to infants fed with infant formula. Additionally, breast fed infant have a different gut microbiota profile as compared to infants fed with infant formula. Altogether, these factors affect the development of the infant physiology, including metabolism, immunity and overall growth.

It has also been reported that feeding patterns of infants and young children—such as the frequency and the amount of ingested food that may depend on their satiety responsiveness—vary depending on the type of milk consumed but also on the mode of milk delivery. A previous study has reported that from the 6th week of life onwards, formula fed infants had significantly higher feeding volumes than breastfed ones (Sievers et al, “Feeding patterns in breast-fed and formula-fed infants”, 2002). Another study has shown that children who were breastfed during the first year show increased satiety responsiveness during the second year compared to those who were formula fed (A Brown et al, “Breastfeeding during the first year promotes satiety responsiveness in children aged 18-24 months”, 2012). These studies suggest that breast milk might contain some factors inducing satiety. In addition infants who are bottle-fed in early infancy are more likely to empty the bottle or cup in late infancy than those who are fed directly at the breast (Li et al, “Do Infants Fed From Bottles Lack Self-regulation of Milk Intake Compared With Directly Breastfed Infants?”, 2010). However, in some cases breastfeeding is inadequate or unsuccessful for medical reasons or the mother chooses not to breast feed. Infant formula have been developed for these situations. Fortifiers have also been developed to enrich mother's milk or infant formula with specific ingredients.

Glucagon-like-peptide-1 (GLP-1 or GLP1) is an incretin secreted by intestinal L-cells upon nutrient intake. GLP-1 has been shown to slow down gastric emptying (Little T J et al. 2006, PMID: 16492694; Nauck M A et al. 1997, PMID:9374685), as well as to reduce appetite and food intake in both, healthy and obese individuals (Pratley et al. 2008; Orskov et al. 1989; Davis H R et al. 1998; PMID:9545022; Domon-Dell et al. 2002; Drucker 2002; Schusdziarra V et al. 2008, PMID:18281111; Punjabi M et al. 2014; PMID: 24601880). GLP-1 has also been shown to reduce body weight/BMI (Zaccardi F. et al. 2016, PMID:26642233; Kelly A S et al. 2013, PMID:23380890; Kelly A S et al. 2012, PMID:22076596) It has also been shown that GLP-1 provides some advantageous cardiovascular effects (Bose et al. 2005, PMID:15616022; Sokos G G et al. 2006, PMID:17174230).

Increasing GLP-1 secretion is therefore an attractive target to control food intake, limit excessive fat mass accumulation and the associated health conditions.

Two pharmacological approaches have been developed to increase GLP-1 or GLP-1-like activity. The first one is by reducing GLP-1 degradation by inhibiting the enzyme responsible for it (DPP-4i). In addition, several GLP-1 receptor agonists have been used to increase GLP-1 receptor activation. However all these pharmacological approaches are indicated only for adults. More “natural” solutions would be preferred for infants and young children.

Prebiotics such as oligofructose have also been shown to stimulate the GLP-1 gut release (Cani et al., 2005, Phuwamongkolwiwat et al., 2014). However the degree of polymerisation fluctuates widely from a type to another and the induced biological effect can therefore vary greatly. In addition, oligofructose is not naturally present in breast milk.

Some reliable and more “natural” solutions, e.g. with ingredients found in breast milk, would therefore be preferred for an administration to infants or young children.

Alternative solutions more appropriate to infants and young children should therefore be developed.

Human milk oligosaccharides (HMOs) are, collectively, the third largest solid constituents in human milk, after lactose and fat. HMOs usually consist of lactose at the reducing end with a carbohydrate core that often contains a fucose or a sialic acid at the non-reducing end. There are over one hundred milk oligosaccharides that have been isolated and characterized in human milk.

Some compositions using HMO ingredients, such as fucosylated oligosaccharides, lacto-N-tetraose, lacto-N-neotetraose and/or sialylated oligosaccharides, have been described for different health purposes, mainly immune purposes.

However the use of HMOs for the prevention of excessive fat mass accumulation and related diseases like obesity later in life has not been deeply explored yet.

There is clearly a need for developing suitable methods to decrease the incidence of later in life diseases related to excessive fat mass accumulation such as later in life obesity, in infants and young children, and/or to control food intake and/or promote a healthy growth in an infant or a young child.

There is also a need to deliver such health benefits in a manner that is particularly suitable for the young subjects (infants and young children), in a manner that does not involve a classical pharmaceutical intervention as the infants or young children are particularly fragile.

There is a need to deliver such health benefits in the infants or young children in a manner that does not induce side effects and/or in a manner that is easy to deliver, and well accepted by the parents or health care practitioners.

There is also a need to deliver such benefits in a manner that does keep the cost of such delivery reasonable and affordable by most.

### SUMMARY OF THE INVENTION

The present inventors have found that sialylated oligosaccharides increased intracellular calcium release using an in vitro system with endocrine intestinal cells, which is an indicator of their potential GLP-1 secretagogue capacity.

Since GLP-1 is especially known to slow down gastric emptying, reduce appetite and food intake, reduce body weight and to provide advantageous cardiovascular benefits, a nutritional composition comprising at least one sialylated oligosaccharide can be used to control food intake, limit excessive fat mass accumulation and the associated health conditions, and provide a healthy growth in an infant or a young child. It can especially be used to prevent a later in life health disorder related to (due to or associated with) excessive fat accumulation, like overweight, obesity, cardiovascular disorders later in life in an infant or a young child.

In a particularly advantageous embodiment, the nutritional composition according to the present invention comprises 3′-sialyllactose (3′-SL), 6′-sialyllactose (6′-SL) or both.

### FIGURES

FIG. 1 represent mean responses (FIG. 1A) and representative calcium response traces (FIG. 1B) of NCI-H716 cells to stimulation with various concentrations of 3SL, showing a dose dependent activation of NCI-H716 cells. p<0.05 vs Negative control; two-sided t-test.

FIG. 2 represents mean responses of NCI-H716 cells to 3SL, sialic acic, lactose, buffer (negative control) and GRP (positive control), showing that the response to 3SL is specific. *p<0.05 vs Negative control; two-sided t-test.
Abbreviations: Buffer=negative control; Sialic=sialic acid; 3SL=3′-sialyllactose; GRP (Gastrin Releasing Peptide)=positive control

FIG. 3 represent mean responses (FIG. 3A) and representative calcium response traces (FIG. 3B) of NCI-H716 cells to stimulation with various concentrations of 6SL, showing a dose dependent activation of NCI-H716 cells. * p<0.05 vs Negative control; two-sided t-test.

### DETAILED DESCRIPTION OF THE INVENTION

As used herein, the following terms have the following meanings.

The term “infant” means a child under the age of 12 months.

The expression “young child” means a child aged between one and three years, also called toddler.

An “infant or young child born by C-section” means an infant or young child who was delivered by caesarean. It means that the infant or young child was not vaginally delivered.

An “infant or young child vaginally born” means an infant or young child who was vaginally delivered and not delivered by caesarean.

A “preterm” or “premature” means an infant or young child who was not born at term. Generally it refers to an infant or young child born prior 36 weeks of gestation.

The expression “nutritional composition” means a composition which nourishes a subject. This nutritional composition is usually to be taken orally or intravenously. It may include a lipid or fat source, a carbohydrate source and/or a protein source. In a particular embodiment the nutritional composition is a ready-to-drink composition such as a ready-to-drink formula.

In a particular embodiment the composition of the present invention is a hypoallergenic nutritional composition. The expression “hypoallergenic nutritional composition” means a nutritional composition which is unlikely to cause allergic reactions.

In a particular embodiment the nutritional composition of the present invention is a “synthetic nutritional composition”. The expression “synthetic nutritional composition” means a mixture obtained by chemical and/or biological means, which can be chemically identical to the mixture naturally occurring in mammalian milks (i.e. the synthetic nutritional composition is not breast milk).

The expression “infant formula” as used herein refers to a foodstuff intended for particular nutritional use by infants during the first months of life and satisfying by itself the nutritional requirements of this category of person (Article 2(c) of the European Commission Directive 91/321/EEC 2006/141/EC of 22 Dec. 2006 on infant formulae and follow-on formulae). It also refers to a nutritional composition intended for infants and as defined in Codex Alimentarius (Codex STAN 72-1981) and Infant Specialities (incl. Food for Special Medical Purpose). The expression “infant formula” encompasses both “starter infant formula” and “follow-up formula” or “follow-on formula”.

A “follow-up formula” or “follow-on formula” is given from the 6th month onwards. It constitutes the principal liquid element in the progressively diversified diet of this category of person.

The expression “baby food” means a foodstuff intended for particular nutritional use by infants or young children during the first years of life.

The expression “infant cereal composition” means a foodstuff intended for particular nutritional use by infants or young children during the first years of life.

The term “fortifier” refers to liquid or solid nutritional compositions suitable for mixing with breast milk or infant formula.

The expression “weaning period” means the period during which the mother's milk is substituted by other food in the diet of an infant or young child.

The expressions “days/weeks/months/years of life”, “days/weeks/months/years after birth” and “days/weeks/months/years of birth” can be used interchangeably.

The expression “later in life” and “in later life” can be used interchangeably. They refer to effects measured in the individual (infant or young child) after the age of some weeks, some months or some years after birth, such as after the age of 6 months after birth, such as after the age of 8 months after birth, such as after the age of 10 months after birth, such as after the age of 1 year after birth, such as after the age of 2 years, preferably after the age of 4 years, more preferably after the age of 5 years, even more preferably after the age of 7 years after birth, or even more, and as a comparison to average observations for subjects of the same age. Preferably it refers to an effect observed after at least 1 year of life, or after at least 2, 5, 7, 10 or 15 years of life. So the expression “later in life” might refer to an observation during infancy, during childhood, during the adolescent period, or during adulthood. Preferably it refers to an observation during childhood, during the adolescent period, or during adulthood.

The expressions “fat mass accumulation” and “fat accumulation” can be used interchangeably. The expression “excessive fat mass accumulation” refers to abnormal fat mass body amount, e.g. in an amount that can lead to health disorders.

The expressions “reducing excessive fat mass accumulation” and “avoiding excessive fat mass accumulation” refer to a decrease or a limitation of the body fat amount of an individual in order to get a normal or a lower fat mass, e.g. in an amount that does not lead to health disorders.

The expression “health disorder(s)” encompass any health conditions and/or diseases and/or dysfunctions that affect the organism of an individual, including the metabolic ones.

The expressions “preventing a health disorder later in life” or “preventing a later in life health disorder” can be used interchangeably. They mean avoiding that a health disorder (e.g. obesity) occur later in life and/or decreasing the incidence and/or the severity of a health disorder later in life. The prevention occurs “later is life”, so preferably after the termination of the intervention or treatment (i.e. after administration of the nutritional composition according to the invention).

The expression “later in life health disorder related to excessive fat mass accumulation” refers to later in life health disorder due to (so direct link) or associated with (so indirect link) fat excess. It encompasses overweight, obesity and obesity related comorbidities.

“Body mass index” or “BMI” is defined as the value resulting from division of a numerator that is the weight in kilograms by a denominator that is the height in meters, squared. Alternatively, the BMI can be calculated from the weight in pounds as the numerator and the height in inches, squared, as the denominator, with the resultant quotient multiplied by 703. “Overweight” is defined for a human as a BMI between 25 and 30. “Obese” is defined for a human as a BMI greater than 30.

“Obesity related comorbidities” include hypertension, dyslipidemia, sleep apnea, arthritis, hyperuricemia, gall bladder disease, cardiovascular disease, metabolic syndrome and certain types of cancer.

The terms “secretion” and “release” can be used interchangeably.

GLP-1 (or GLP1) means Glucagon-like-peptide-1. It is an incretin secreted by intestinal endocrine cells known as L-cells. The expressions “increasing GLP-1 secretion” and “increasing GLP-1 release” can be used interchangeably. They mean that the amount of GLP-1 secreted for example by the intestinal epithelium, such as in the ileum or colon, among others, is higher in an individual fed with the nutritional composition according to the present invention (i.e. comprising at least one sialylated oligosaccharide) in comparison with a standard composition (i.e. a nutritional composition not comprising at least one sialylated oligosaccharide). In a particular embodiment, the expression “increasing GLP-1 secretion” refers to “increasing intestinal GLP-1 secretion”.

The GLP-1 secretion/release may be measured by techniques known by the skilled person such as by measuring its amount in blood circulation (i.e. by determination of plasma GLP-1 concentration) of an individual since GLP-1 is secreted into the bloodstream upon nutrient intake.

The expression “reducing and/or controlling food intake” means that the amount of food ingested by the infant or young children when eating the nutritional composition of the present invention (i.e. comprising at least one sialylated oligosaccharide) will be reduced or regulated so that it gets lower than when eating a standard nutritional composition (i.e. not comprising at least one sialylated oligosaccharide). In some embodiments the ingested amount of the nutritional composition of the present invention gets closer or approximates to the amount ingested for breastfeeding. The intake or amount may refer to the quantity per meal or per day.

The term “growth” refers to growth in weight, height and/or head circumference of an infant or young child. In a particular embodiment it refers to the weight. The growth has to be understood as the evolution of the weight, height and/or head circumference over the aging of the infant or young child. These parameters do not exclusively increase during development of the infant, as indeed the standard curves of growth published by the WHO show that the weight of an infant may decrease in the first days of life of the infant. Therefore, the growth has to be understood as the overall growth of the infant over the first months of life. So the expressions “growth rate” and “rate of growth” can also be used alternatively to the term “growth”.

The expressions “promoting a healthy growth” and “promoting an optimal growth” can be used interchangeably. They encompasses promoting a rate of growth which gets closer or approximates to the rate of growth of a breast-fed infant. They encompass promoting a growth that is qualified as normal by pediatricians so that it is not associated with providing health issues. These expressions also encompass preventing excessive growth or excessive body weight gain that may occur in formula-fed infants, especially in the first few months of life. The expression “promoting a healthy growth” may also encompass controlling weight management and/or avoiding weight gain, especially excessive weight gain, and/or promoting a lean mass increase (especially over a total weight or adipose mass increase).

“Satiety” is the feeling of fullness after eating that suppresses the urge to eat for a period of time after a meal. The expression “increasing the satiety responsiveness” (or “inducing satiety”) encompasses getting satiety earlier in time (i.e. faster) in an infant or young child administered the nutritional composition of the present invention (i.e. comprising at least one sialylated oligosaccharide) in comparison to an infant or young child administered a conventional nutritional composition (i.e. not comprising at least one sialylated oligosaccharide), i.e. less amount of food will be ingested in order for the infant or young child to feel fullness. It may also mean “regulating (e.g. decreasing/lowering) appetite”. Satiety may be reached at a time that gets closer or that approximates to the time obtained when breastfeeding.

The “mother's milk” should be understood as the breast milk or the colostrum of the mother.

The term “HMO” or “HMOs” refers to human milk oligosaccharide(s). These carbohydrates are resistant to enzymatic hydrolysis by digestive enzymes (e.g pancreatic and/or brush border), indicating that they may display functions not directly related to their caloric value. It has especially been illustrated that they play a vital role in the early development of infants and young children, such as the maturation of the immune system. Many different kinds of HMOs are found in the human milk. Each individual oligosaccharide is based on a combination of glucose, galactose, sialic acid (N-acetylneuraminic acid), fucose and/or N-acetylglucosamine with many and varied linkages between them, thus accounting for the enormous number of different oligosaccharides in human milk—over 130 such structures have been identified so far. Almost all of them have a lactose moiety at their reducing end while sialic acid and/or fucose (when present) occupy terminal positions at the non-reducing ends. The HMOs can be acidic (e.g. charged sialic acid containing oligosaccharide) or neutral (e.g. fucosylated oligosaccharide). Some examples of HMOs are the fucosylated oligosaccharides, the N-acetylated oligosaccharides and/or the sialylated oligosaccharides.

A “sialylated oligosaccharide” is a charged sialic acid containing oligosaccharide, i.e. an oligosaccharide having a sialic acid residue. It has an acidic nature. Some examples are 3-SL (3′ sialyllactose) and 6-SL (6′ sialyllactose). The expressions “sialylated oligosaccharide” and “sialyllactose (SL)” can be used interchangeably. The trisaccharide sialyllactose consists of lactose at the reducing terminus and one sialic acid residue at the non-reducing end via an α-2,3 binding or α-2,6 binding, resulting in 3′-sialyllactose (3′-SL) and 6′-sialyllactose (6′-SL), respectively.

In the context of the present disclosure, “3′-sialyllactose” (3′-SL, 3-SL, 3′SL, or 3SL) refers to (6R)-5-Acetamido-3,5-dideoxy-6-[(1R,2R)-1,2,3-trihydroxypropyl]-β-L-threo-hex-2-ulopyranonosyl-(2->3)β-D-galactopyranosyl-(1->4)-D-glucopyranose (IUPAC), and “6′-sialyllactose” (6′-SL, 6-SL, 6′SL, or 6SL) refers to (6R)-5-Acetamido-3,5-dideoxy-6-[(1R,2R)-1,2,3-trihydroxypropyl]-β-L-threo-hex-2-ulopyranonosyl-(2->6)β-D-galactopyranosyl-(1->4)-D-glucopyranose (IUPAC).

A “fucosylated oligosaccharide” is an oligosaccharide having a fucose residue. It has a neutral nature. Some examples are 2′-FL (2′-fucosyllactose or 2-fucosyllactose or 2FL or 2-FL), 3-FL (3-fucosyllactose), difucosyllactose, lacto-N-fucopentaose (e.g. lacto-N-fucopentaose I, lacto-N-fucopentaose II, lacto-N-fucopentaose III, lacto-N-fucopentaose V), lacto-N-fucohexaose, lacto-N-difucohexaose I, fucosyllacto-N-hexaose, fucosyllacto-N-neohexaose, difucosyllacto-N-hexaose I, difucosyllacto-N-neohexaose II and any combination thereof.

The expressions “fucosylated oligosaccharides comprising a 2′-fucosyl-epitope” and “2-fucosylated oligosaccharides” encompass fucosylated oligosaccharides with a certain homology of form since they contain a 2′-fucosyl-epitope, therefore a certain homology of function can be expected.

The expression “N-acetylated oligosaccharide(s)” encompasses both “N-acetyl-lactosamine” and “oligosaccharide(s) containing N-acetyl-lactosamine”. They are neutral oligosaccharides having an N-acetyl-lactosamine residue. Suitable examples are LNT (lacto-N-tetraose), para-lacto-N-neohexaose (para-LNnH), LNnT (lacto-N-neotetraose) or any combination thereof. Other examples are lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose.

A “precursor of HMO” is a key compound that intervenes in the manufacture of HMO, such as sialic acid and/or fucose.

The expressions “galacto-oligosaccharide”, “galactooligosaccharide” and “GOS” can be used interchangeably. They refer to an oligosaccharide comprising two or more galactose molecules which has no charge and no N-acetyl residue (i.e. they are neutral oligosaccharide). In a particular embodiment, said two or more galactose molecules are linked by a β-1,2, β-1,3, β-1,4 or β-1,6 linkage. In another embodiment, “galacto-oligosaccharide” and “GOS” also include oligosaccharides comprising one galactose molecule and one glucose molecule (i.e. disaccharides) which are linked by a β-1,2, β-1,3 or β-1,6 linkage.

The nutritional composition of the present invention can be in solid form (e.g. powder) or in liquid form. The amount of the various ingredients (e.g. the oligosaccharides) can be expressed in g/100 g of composition on a dry weight basis when it is in a solid form, e.g. a powder, or as a concentration in g/L of the composition when it refers to a liquid form (this latter also encompasses liquid composition that may be obtained from a powder after reconstitution in a liquid such as milk, water . . . , e.g. a reconstituted infant formula or follow-on/follow-up formula or infant cereal product or any other formulation designed for infant nutrition).

The term “prebiotic” means non-digestible carbohydrates that beneficially affect the host by selectively stimulating the growth and/or the activity of healthy bacteria such as bifidobacteria in the colon of humans (Gibson G R, Roberfroid M B. Dietary modulation of the human colonic microbiota: introducing the concept of prebiotics. J Nutr. 1995; 125:1401-12).

The term “probiotic” means microbial cell preparations or components of microbial cells with a beneficial effect on the health or well-being of the host. (Salminen S, Ouwehand A. Benno Y. et al. “Probiotics: how should they be defined” Trends Food Sci. Technol. 1999:10 107-10). The microbial cells are generally bacteria or yeasts.

The term “cfu” should be understood as colony-forming unit.

All percentages are by weight unless otherwise stated.

In addition, in the context of the invention, the terms “comprising” or “comprises” do not exclude other possible elements. The composition of the present invention, including the many embodiments described herein, can comprise, consist of, or consist essentially of the essential elements and limitations of the invention described herein, as well as any additional or optional ingredients, components, or limitations described herein or otherwise depending on the needs. Any reference to prior art documents in this specification is not to be considered an admission that such prior art is widely known or forms part of the common general knowledge in the field.

The invention will now be described in further details. It is noted that the various aspects, features, examples and embodiments described in the present application may be compatible and/or combined together.

The present invention therefore refers to a nutritional composition comprising at least one sialylated oligosaccharide, for use in reducing and/or avoiding excessive fat mass accumulation of an infant or a young child, especially later in life excessive fat mass accumulation.

This nutritional composition can also be used for preventing a later in life health disorder related to excessive fat mass accumulation in an infant or a young child, more particularly later in life obesity or a related comorbidity (i.e. a later in life obesity related comorbidity) selected from the list consisting of hypertension, dyslipidemia, sleep apnea, arthritis, hyperuricemia, gall bladder disease, cardiovascular disease and metabolic syndrome.

In a preferred embodiment, the nutritional composition of the present invention is for use in preventing obesity later in life in an infant or a young child.

It can be also used for reducing the risk of development of overweight later in life in an infant or a young child.

The present invention also refers to a nutritional composition comprising at least one sialylated oligosaccharide, for use in reducing and/or controlling food intake in an infant or a young child and/or for use in promoting a healthy growth in an infant or a young child.

The present inventors have indeed found that sialylated oligosaccharides (both 3SL and 6SL) increased intracellular calcium release using an in vitro system with endocrine intestinal cells, which is an indicator of their potential GLP-1 secretagogue capacity, as deeper explained in the experimental part. Indeed, the release of GLP-1 by intestinal L-cells is dependent on the intracellular release of Ca2+. As shown in Example 2, sialylated oligosaccharides effectively increased intracellular calcium concentration. Without being bound by theory, the inventors of the present invention believe that these particular oligosaccharides would therefore significantly increase the GLP-1 secretion of an individual and therefore be useful in providing the above-mentioned health benefits associated thereof.

The nutritional composition according to the present invention comprises sialylated oligosaccharide(s). There can be one or several sialylated oligosaccharide(s), i.e. one or several type(s)/category(ies) of sialylated oligosaccharide(s). The sialylated oligosaccharide(s) is preferably selected from the group consisting of 3′ sialyllactose (3-SL), 6′ sialyllactose (6-SL), and any combination thereof.

In particularly advantageous embodiments, the nutritional composition according to the present invention comprises 3-SL.

In some embodiments of the invention the nutritional composition according to the present invention comprises 6-SL.

In some embodiments of the invention the nutritional composition comprises 3-SL and 6-SL. In some particular embodiments the ratio between 3′-sialyllactose (3-SL) and 6′-sialyllactose (6-SL) can be in the range between 5:1 and 1:10, or from 3:1 and 1:1, or from 1:1 to 1:10.

In some specific embodiments the sialylated oligosaccharide of the composition is only 3′ sialyllactose (3-SL), i.e. the nutritional composition according to the invention comprises sialylated oligosaccharide that consists of 3-SL.

The sialylated oligosaccharide(s) may be isolated by chromatographic or filtration technology from a natural source such as animal milks. Alternatively, they may be produced by biotechnological means using specific sialyltransferases or sialidases, neuraminidases, either by an enzyme based fermentation technology (recombinant or natural enzymes), by chemical synthesis or by a microbial fermentation technology. In the latter case microbes may either express their natural enzymes and substrates or may be engineered to produce respective substrates and enzymes. Single microbial cultures or mixed cultures may be used. Sialyl-oligosaccharide formation can be initiated by acceptor substrates starting from any degree of polymerisation (DP), from DP=1 onwards. Alternatively, sialyllactoses may be produced by chemical synthesis from lactose and free N′-acetylneuraminic acid (sialic acid). Sialyllactoses are also commercially available for example from Kyowa Hakko Kogyo of Japan.

The nutritional composition according to the present invention comprises sialylated oligosaccharide(s) that may be present in a total amount of from 0.1 to 10 wt %, such as from 0.5 to 7 wt % or from 1 to 5 wt % of the nutritional composition before reconstitution with water. For reconstituted ready-to-drink formula, the amount could be from 0.01 to 1%, more preferably 0.05 to 0.7% or 0.1 to 0.5%.

In particular embodiments, the nutritional composition of the present invention may comprise sialylated oligosaccharide(s) in a total amount of from 0.05 to 5 g/L of, for example from 0.1 to 4 g/L, or from 0.3 to 2 g/L of the composition, or in a total amount of from 0.03 to 3.5 g/100 g, for example from 0.1 to 2 g or from 0.2 to 1 g/100 g of composition on a dry weight basis.

The nutritional composition according to the present invention may also comprise at least another oligosaccharide(s) (i.e. other than the sialylated oligosaccharide necessarily present in the composition) and/or at least a fiber(s) and/or at least a precursor(s) of human milk oligosaccharide(s). The other oligosaccharide and/or fiber and/or precursor may be selected from the list consisting of fucosylated oligosaccharide(s), N-acetylated oligosaccharide(s), galacto-oligosaccharides (GOS), fructo-oligosaccharides (FOS), inulin, xylooligosaccharides (XOS), polydextrose, sialic acid, fucose and any combination thereof. They may be in an amount between 0 and 10% by weight of composition.

For example, the nutritional composition of the present invention may also comprise at least one fucosylated oligosaccharide. There can be one or several types of fucosylated oligosaccharide(s). The fucosylated oligosaccharide(s) can indeed be selected from the list comprising 2′-fucosyllactose, 3′fucosyllactose, difucosyllactose, lacto-N-fucopentaose (such as lacto-N-fucopentaose I, lacto-N-fucopentaose II, lacto-N-fucopentaose III, lacto-N-fucopentaose V), lacto-N-fucohexaose, lacto-N-difucohexaose I, fucosyllacto-N-hexaose, fucosyllacto-N-neohexaose (such as fucosyllacto-N-neohexaose I, fucosyllacto-N-neohexaose II), difucosyllacto-N-hexaose I, difuco-lacto-N-neohexaose, difucosyllacto-N-neohexaose I, difucosyllacto-N-neohexaose II, fucosyl-para-Lacto-N-hexaose, tri-fuco-para-Lacto-N-hexaose I and any combination thereof.

In some particular embodiments the fucosylated oligosaccharide comprises a 2′-fucosyl-epitope. It can be for example selected from the list comprising 2′-fucosyllactose, difucosyllactose, lacto-N-fucopentaose, lacto-N-fucohexaose, lacto-N-difucohexaose, fucosyllacto-N-hexaose, fucosyllacto-N-neohexaose, difucosyllacto-N-hexaose difuco-lacto-N-neohexaose, difucosyllacto-N-neohexaose, fucosyl-para-Lacto-N-hexaose and any combination thereof.

In some particular embodiments, the nutritional composition according to the invention may comprise 2′-fucosyllactose (or 2FL, or 2′FL, or 2-FL or 2′-FL).

The fucosylated oligosaccharide(s) may be isolated by chromatography or filtration technology from a natural source such as animal milks. Alternatively, it may be produced by biotechnological means using specific fucosyltransferases and/or fucosidases either through the use of enzyme-based fermentation technology (recombinant or natural enzymes) or microbial fermentation technology. In the latter case, microbes may either express their natural enzymes and substrates or may be engineered to produce respective substrates and enzymes. Single microbial cultures and/or mixed cultures may be used. Fucosylated oligosaccharide formation can be initiated by acceptor substrates starting from any degree of polymerization (DP), from DP=1 onwards. Alternatively, fucosylated oligosaccharides may be produced by chemical synthesis from lactose and free fucose. Fucosylated oligosaccharides are also available for example from Kyowa, Hakko, Kogyo of Japan.

As another example, the nutritional composition of the present invention may also comprise at least one the N-acetylated oligosaccharide. There can be one or several types of N-acetylated oligosaccharide. The N-acetylated oligosaccharide(s) can be for example lacto-N-tetraose (LNT), lacto-N-neotetraose (LNnT) or any combination thereof. In some particular embodiments the composition comprises both LNT and LNnT in a ratio LNT:LNnT between 5:1 and 1:2, or from 2:1 to 1:1, or from 2:1.2 to 2:1.6.

The N-acetylated oligosaccharide(s) may be synthesised chemically by enzymatic transfer of saccharide units from donor moieties to acceptor moieties using glycosyltransferases as described for example in U.S. Pat. No. 5,288,637 and WO 96/10086. Alternatively, LNT and LNnT may be prepared by chemical conversion of Keto-hexoses (e.g. fructose) either free or bound to an oligosaccharide (e.g. lactulose) into N-acetylhexosamine or an N-acetylhexosamine-containing oligosaccharide as described in Wrodnigg, T. M.; Stutz, A. E. (1999) Angew. Chem. Int. Ed. 38:827-828. N-acetyl-lactosamine produced in this way may then be transferred to lactose as the acceptor moiety. The N-acetylated oligosaccharide(s) may also be produced by biotechnological means based on microbial fermentation technology.

In a particular embodiment of the present invention, the nutritional composition comprises 2′-fucosyllactose (2FL) and lacto-N-neotetraose (LNnT) in addition to the sialylated oligosaccharide(s), e.g. in a 2FL:LNnT weight ratio is from 1:10 to 12:1, such as from 1:7 to 10:1 or from 1:5 to 5:1 or from 2:1 to 5:1 or from 1:3 to 3:1, or from 1:2 to 2:1, or from 1:1 to 3:1, or from 1:5 to 1:0.5; for example 2:1 or 10:1.

The nutritional composition of the present invention may for example comprise:
fucosylated oligosaccharide(s) in a total amount of 0.2-5 g/L, for example 0.5-4.5 g/L or 1-4 g/L of the composition, or in a total amount of 0.13-3.48 g/100 g, for example 0.34-3.13 g/100 g or 0.69-2.78 g/100 g of composition on a dry weight basis; and/or
N-acetylated oligosaccharide(s) in a total amount of 0.05-5 g/L, for example 0.1-2 g/L or 0.1-1 g/L of the composition, or in a total amount of 0.0.03-3.48 g/100 g, for example 0.07-1.4 g/100 g or 0.07-0.7 g/100 g of composition on a dry weight basis.

In some other particular embodiments, in addition to the sialylated oligosaccharide(s), the nutritional composition of the present invention comprises at least another oligosaccharide selected from the list consisting of 2′-fucosyllactose (2FL), 3′fucosyllactose (3FL), difucosyllactose (DFL), lacto-N-tetraose (LNT), lacto-N-neotetraose (LNnT) and any combination thereof

The nutritional composition of the present invention may also comprise FOS and/or inulin. Suitable commercial products that can be used in addition to the sialylated oligosaccharides comprised in the nutritional compositions according to the invention include combinations of FOS with inulin such as the product sold by BENEO under the trademark Orafti, or polydextrose sold by Tate & Lyle under the trademark STA-LITE®.

The nutritional composition according to the present invention may also comprise GOS or mixture comprising GOS.

In a particular embodiment, the nutritional composition can also contain at least one BMO (bovine milk oligosaccharide).

In a particular embodiment, the nutritional composition may additionally comprise an oligosaccharide mixture (“BMOS”) that comprises from 0.1 to 4.0 wt % of N-acetylated oligosaccharide(s), from 92.0 to 99.5 wt % of the galacto-oligosaccharide(s) and from 0.2 to 4.0 wt % of the sialylated oligosaccharide(s). WO2006087391 and WO2012160080 provide some examples of production of a BMOS mixture.

The nutritional composition according to the present invention may optionally also comprise at least one precursor of human milk oligosaccharide. There can be one or several precursor(s). For example the precursor of human milk oligosaccharide is sialic acid, fucose or a mixture thereof. In some particular embodiments the composition comprises sialic acid.

In particular examples the composition comprises from 0 to 3 g/L of precursor(s) of human milk oligosaccharide, or from 0 to 2 g/L, or from 0 to 1 g/L, or from 0 to 0.7 g/L, or from 0 to 0.5 g/L or from 0 to 0.3 g/L, or from 0 to 0.2 g/L of precursor(s) of human milk oligosaccharide. The composition according to the invention can contain from 0 to 2.1 g of precursor(s) of human milk oligosaccharide per 100 g of composition on a dry weight basis, e.g. from 0 to 1.5 g or from 0 to 0.8 g or from 0 to 0.15 g of precursor(s) of human milk oligosaccharide per 100 g of composition on a dry weight basis.

The nutritional composition of the present invention can further comprise at least one probiotic (or probiotic strain), such as a probiotic bacterial strain.

The probiotic microorganisms most commonly used are principally bacteria and yeasts of the following genera: Lactobacillus spp., Streptococcus spp., Enterococcus spp., Bifidobacterium spp. and Saccharomyces spp.

In some particular embodiments, the probiotic is a probiotic bacterial strain. In some specific embodiments, it is particularly Bifidobacteria and/or Lactobacilli.

Suitable probiotic bacterial strains include Lactobacillus rhamnosus ATCC 53103 available from Valio Oy of Finland under the trademark LGG, Lactobacillus rhamnosus CGMCC 1.3724, Lactobacillus paracasei CNCM 1-2116, Lactobacillus johnsonii CNCM 1-1225, Streptococcus salivarius DSM 13084 sold by BLIS Technologies Limited of New Zealand under the designation K12, Bifidobacterium lactis CNCM 1-3446 sold inter alia by the Christian Hansen company of Denmark under the trademark Bb 12, Bifidobacterium longum ATCC BAA-999 sold by Morinaga Milk Industry Co. Ltd. of Japan under the trademark BB536, Bifidobacterium breve sold by Danisco under the trademark Bb-03, Bifidobacterium breve sold by Morinaga under the trade mark M-16V, Bifidobacterium infantis sold by Procter & Gamble Co. under the trademark Bifantis and Bifidobacterium breve sold by Institut RoseII (Lallemand) under the trademark R0070.

The nutritional composition according to the invention may contain from 10e3 to 10e12 cfu of probiotic strain, more preferably between 10e7 and 10e12 cfu such as between 10e8 and 10e10 cfu of probiotic strain per g of composition on a dry weight basis.

In one embodiment the probiotics are viable. In another embodiment the probiotics are non-replicating or inactivated. There may be both viable probiotics and inactivated probiotics in some other embodiments.

The nutritional composition of the invention can further comprise at least one phage (bacteriophage) or a mixture of phages, preferably directed against pathogenic Streptococci, Haemophilus, Moraxella and Staphylococci.

The nutritional composition according to the invention can be for example an infant formula, a starter infant formula, a follow-on or follow-up formula, a baby food, an infant cereal composition, a fortifier such as a human milk fortifier, or a supplement. In some particular embodiments, the composition of the invention is an infant formula, a fortifier or a supplement that may be intended for the first 4 or 6 months of age. In a preferred embodiment the nutritional composition of the invention is an infant formula.

In some other embodiments the nutritional composition of the present invention is a fortifier. The fortifier can be a breast milk fortifier (e.g. a human milk fortifier) or a formula fortifier such as an infant formula fortifier or a follow-on/follow-up formula fortifier.

When the nutritional composition is a supplement, it can be provided in the form of unit doses.

The nutritional composition of the present invention can be in solid (e.g. powder), liquid or gelatinous form.

The nutritional composition according to the invention generally contains a protein source. The protein can be in an amount of from 1.5 to 3 g per 100 kcal. In some embodiments, especially when the composition is intended for premature infants, the protein amount can be between 2.4 and 4 g/100 kcal or more than 3.6 g/100 kcal. In some other embodiments the protein amount can be below 2.0 g per 100 kcal, e.g. between 1.8 to 2 g/100 kcal, or in an amount below 1.8 g per 100 kcal.

The type of protein is not believed to be critical to the present invention provided that the minimum requirements for essential amino acid content are met and satisfactory growth is ensured. Thus, protein sources based on whey, casein and mixtures thereof may be used as well as protein sources based on soy. As far as whey proteins are concerned, the protein source may be based on acid whey or sweet whey or mixtures thereof and may include alpha-lactalbumin and beta-lactoglobulin in any desired proportions.

In some advantageous embodiments the protein source is whey predominant (i.e. more than 50% of proteins are coming from whey proteins, such as 60% or 70%).

The proteins may be intact or hydrolysed or a mixture of intact and hydrolysed proteins. By the term “intact” is meant that the main part of the proteins are intact, i.e. the molecular structure is not altered, for example at least 80% of the proteins are not altered, such as at least 85% of the proteins are not altered, preferably at least 90% of the proteins are not altered, even more preferably at least 95% of the proteins are not altered, such as at least 98% of the proteins are not altered. In a particular embodiment, 100% of the proteins are not altered.

The term “hydrolysed” means in the context of the present invention a protein which has been hydrolysed or broken down into its component amino acids.

The proteins may be either fully or partially hydrolysed. It may be desirable to supply partially hydrolysed proteins (degree of hydrolysis between 2 and 20%), for example for infants or young children believed to be at risk of developing cow's milk allergy. If hydrolysed proteins are required, the hydrolysis process may be carried out as desired and as is known in the art. For example, whey protein hydrolysates may be prepared by enzymatically hydrolysing the whey fraction in one or more steps. If the whey fraction used as the starting material is substantially lactose free, it is found that the protein suffers much less lysine blockage during the hydrolysis process. This enables the extent of lysine blockage to be reduced from about 15% by weight of total lysine to less than about 10% by weight of lysine; for example about 7% by weight of lysine which greatly improves the nutritional quality of the protein source.

In an embodiment of the invention at least 70% of the proteins are hydrolysed, preferably at least 80% of the proteins are hydrolysed, such as at least 85% of the proteins are hydrolysed, even more preferably at least 90% of the proteins are hydrolysed, such as at least 95% of the proteins are hydrolysed, particularly at least 98% of the proteins are hydrolysed. In a particular embodiment, 100% of the proteins are hydrolysed.

In one particular embodiment the proteins of the nutritional composition are hydrolyzed, fully hydrolyzed or partially hydrolyzed. The degree of hydrolysis (DH) of the protein can be between 8 and 40, or between 20 and 60 or between 20 and 80 or more than 10, 20, 40, 60, 80 or 90.

In a particular embodiment the nutritional composition according to the invention is a hypoallergenic composition. In another particular embodiment the composition according to the invention is a hypoallergenic nutritional composition.

The nutritional composition according to the present invention generally contains a carbohydrate source. This is particularly preferable in the case where the nutritional composition of the invention is an infant formula. In this case, any carbohydrate source conventionally found in infant formulae such as lactose, sucrose, saccharose, maltodextrin, starch and mixtures thereof may be used although one of the preferred sources of carbohydrates is lactose.

The nutritional composition according to the present invention generally contains a source of lipids. This is particularly relevant if the nutritional composition of the invention is an infant formula. In this case, the lipid source may be any lipid or fat which is suitable for use in infant formulae. Some suitable fat sources include palm oil, high oleic sunflower oil and high oleic safflower oil. The essential fatty acids linoleic and α-linolenic acid may also be added, as well small amounts of oils containing high quantities of preformed arachidonic acid and docosahexaenoic acid such as fish oils or microbial oils. The fat source may have a ratio of n-6 to n-3 fatty acids of about 5:1 to about 15:1; for example about 8:1 to about 10:1.

The nutritional composition of the invention may also contain all vitamins and minerals understood to be essential in the daily diet and in nutritionally significant amounts. Minimum requirements have been established for certain vitamins and minerals. Examples of minerals, vitamins and other nutrients optionally present in the composition of the invention include vitamin A, vitamin B1, vitamin B2, vitamin B6, vitamin B12, vitamin E, vitamin K, vitamin C, vitamin D, folic acid, inositol, niacin, biotin, pantothenic acid, choline, calcium, phosphorous, iodine, iron, magnesium, copper, zinc, manganese, chlorine, potassium, sodium, selenium, chromium, molybdenum, taurine, and L-carnitine. Minerals are usually added in salt form. The presence and amounts of specific minerals and other vitamins will vary depending on the intended population.

If necessary, the nutritional composition of the invention may contain emulsifiers and stabilisers such as soy, lecithin, citric acid esters of mono- and diglycerides, and the like.

The nutritional composition of the invention may also contain other substances which may have a beneficial effect such as lactoferrin, nucleotides, nucleosides, and the like.

The nutritional composition of the invention may also contain carotenoid(s). In some particular embodiments of the invention, the nutritional composition of the invention does not comprise any carotenoid.

The nutritional composition according to the invention may be prepared in any suitable manner. A composition will now be described by way of example.

For example, a formula such as an infant formula may be prepared by blending together the protein source, the carbohydrate source and the fat source in appropriate proportions. If used, the emulsifiers may be included at this point. The vitamins and minerals may be added at this point but they are usually added later to avoid thermal degradation. Any lipophilic vitamins, emulsifiers and the like may be dissolved into the fat source prior to blending. Water, preferably water which has been subjected to reverse osmosis, may then be mixed in to form a liquid mixture. The temperature of the water is conveniently in the range between about 50° C. and about 80° C. to aid dispersal of the ingredients. Commercially available liquefiers may be used to form the liquid mixture.

The sialylated oligosaccharide(s) may be added at this stage, especially if the final product is to have a liquid form. If the final product is to be a powder, they may likewise be added at this stage if desired.

The liquid mixture is then homogenised, for example in two stages.

The liquid mixture may then be thermally treated to reduce bacterial loads, by rapidly heating the liquid mixture to a temperature in the range between about 80° C. and about 150° C. for a duration between about 5 seconds and about 5 minutes, for example. This may be carried out by means of steam injection, an autoclave or a heat exchanger, for example a plate heat exchanger.

Then, the liquid mixture may be cooled to between about 60° C. and about 85° C. for example by flash cooling. The liquid mixture may then be again homogenised, for example in two stages between about 10 MPa and about 30 MPa in the first stage and between about 2 MPa and about 10 MPa in the second stage. The homogenised mixture may then be further cooled to add any heat sensitive components, such as vitamins and minerals. The pH and solids content of the homogenised mixture are conveniently adjusted at this point.

If the final product is to be a powder, the homogenised mixture is transferred to a suitable drying apparatus such as a spray dryer or freeze dryer and converted to powder. The powder should have a moisture content of less than about 5% by weight. The sialylated oligosaccharide(s) may also or alternatively be added at this stage by dry-mixing or by blending them in a syrup form of crystals, along with the probiotic strain(s) (if used), and the mixture is spray-dried or freeze-dried.

If a liquid composition is preferred, the homogenised mixture may be sterilised then aseptically filled into suitable containers or may be first filled into the containers and then retorted.

In another embodiment, the composition of the invention may be a supplement.

The supplement may be in the form of tablets, capsules, pastilles or a liquid for example. The supplement may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents and gel forming agents. The supplement may also contain conventional pharmaceutical additives and adjuvants, excipients and diluents, including, but not limited to, water, gelatine of any origin, vegetable gums, lignin-sulfonate, talc, sugars, starch, gum arabic, vegetable oils, polyalkylene glycols, flavouring agents, preservatives, stabilizers, emulsifying agents, buffers, lubricants, colorants, wetting agents, fillers, and the like.

Further, the supplement may contain an organic or inorganic carrier material suitable for oral or parenteral administration as well as vitamins, minerals trace elements and other micronutrients in accordance with the recommendations of Government bodies such as the USRDA.

The nutritional composition according to the invention is for use in infants or young children. The infants or young children may be born term or preterm. In a particular embodiment the nutritional composition of the invention is for use in infants or young children that were born preterm. Preterm infants may be at increased risk of poor nutrient utilization, impaired lean body mass growth, fat accumulation in the visceral area and metabolic disease later in life. So in a particular embodiment the nutritional composition of the invention is for use in preterm infants.

The nutritional composition of the present invention may also be used in an infant or a young child that was born by C-section or that was vaginally delivered.

In some embodiments the nutritional composition according to the invention can be for use before and/or during the weaning period.

In some embodiments the nutritional composition according to the invention is for use in infants or young children at risk and/or in need.

In some embodiments the nutritional composition according to the invention is for use in infants or young children at risk of developing a later in life health disorder related to excessive fat mass accumulation. Infants or young children at risk of developing later in life overweight or obesity may be targeted. In some embodiments the nutritional composition of the present invention is for use in infants or young children born from overweight and obese women. Indeed, scientific evidence continues to suggest that infants born to overweight and obese mothers have a greater risk of becoming overweight or obese later in life than infants born to mothers who are not overweight or obese. In some embodiments the nutritional composition of the present invention is for use in infants or young children born from mothers who experienced gestational diabetes.

The infants or young children at risk and/or in need may be bottle-fed and/or formula-fed infants or young children.

The infants or young children at risk and/or in need may be infants or young children who meet at least one of the following criteria:
they have difficulties with controlling their food intake or they have an abnormal (especially higher) food intake (e.g. they have a higher appetite), for example:
   i) they eat more than other infants or young children of the same age and of the same size (weight and length); and/or
   ii) they have an altered perception of satiety, for example a delayed satiety responsiveness (that might be due to physiological or anatomical reasons); and/or
they have an excessive weight gain during the first few months of life.

In a particular example, the nutritional composition of the present invention may be used in infants or young children who were IUGR (intrauterine growth restricted). This particular population is at risk and/or in need since they will have a higher appetite to compensate their growth retardation. But they may not eat in a healthy way with standard formulations, e.g. they may have a higher total weight or adipose mass increase over the lean mass increase, which may lead to the development and programming for future health conditions including later in life obesity or future related comorbidities. The nutritional composition of the present invention is believed to provide a healthy growth.

The nutritional composition can be administered (or given or fed) at an age and for a period that depends on the possibilities and needs.

The nutritional composition may be used for prevention purposes and/or for treatment purposes.

The nutritional composition can for example be given immediately after birth of the infants, especially when it is used for prevention purposes. The composition of the invention can also be given during the first week of life of the infant, or during the first 2 weeks of life, or during the first 3 weeks of life, or during the first month of life, or during the first 2 months of life, or during the first 3 months of life, or during the first 4 months of life, or during the first 6 months of life, or during the first 8 months of life, or during the first 10 months of life, or during the first year of life, or during the first two years of life or even more. In some particularly advantageous embodiments of the invention, the nutritional composition is given (or administered) to an infant within the first 4 or 6 months of birth of said infant. In some other embodiments, the nutritional composition of the invention is given few days (e.g. 1, 2, 3, 5, 10, 15, 20 . . . ), or few weeks (e.g. 1, 2, 3, 4, 5, 6, 7, 8, 9, 10 . . . ), or few months (e.g. 1, 2, 3, 4, 5, 6, 7, 8, 9, 10 . . . ) after birth. This may be especially the case when the infant is premature, but not necessarily.

For treatment purposes, the composition may be given once symptoms appear, e.g. when a mother or a paediatrician discovers that the infant or young child is too often hungry and/or needs abnormal (higher) food amounts before reaching satiety and/or has an excessive growth/weight gain especially during the first few months of life. It may be given up to the symptoms disappear, or several days/weeks/months after said disappearance.

In one embodiment the composition of the invention is given to the infant or young child as a supplementary composition to the mother's milk. In some embodiments the infant or young child receives the mother's milk during at least the first 2 weeks, first 1, 2, 4, or 6 months. In one embodiment the nutritional composition of the invention is given to the infant or young child after such period of mother's nutrition, or is given together with such period of mother's milk nutrition. In another embodiment the composition is given to the infant or young child as the sole or primary nutritional composition during at least one period of time, e.g. after the 1^{st}, 2^{nd} or 4^{th} month of life, during at least 1, 2, 4 or 6 months.

In one embodiment the nutritional composition of the invention is a complete nutritional composition (fulfilling all or most of the nutritional needs of the subject). In another embodiment the nutrition composition is a supplement or a fortifier intended for example to supplement human milk or to supplement an infant formula or a follow-on formula.

As illustrated in the experimental part, the present inventors have found that sialylated oligosaccharides increased intracellular calcium release using an in vitro system with endocrine intestinal cells, which is an indicator of their potential GLP-1 secretagogue capacity. As previously mentioned, GLP-1 is especially known to slow down gastric emptying, reduce appetite, reduce food intake, reduce body weight and to provide advantageous cardiovascular benefits.

The nutritional composition according to the present invention would therefore be useful in reducing and/or avoiding excessive fat mass accumulation of an infant or a young child, especially excessive fat mass accumulation in later life. It may also be used in preventing later in life health disorders related to excessive fat mass accumulation in an infant or young child, especially later in life obesity or obesity related comorbidities.

In a particular embodiment, the nutritional composition according to the present invention may be used for preventing any later in life health disorder related to excessive fat mass accumulation selected from the list consisting of overweight, obesity or obesity related comorbidities.

Some examples of later in life obesity related comorbidities are: cardiovascular disease, hypertension, dyslipidemia, sleep apnea, arthritis, hyperuricemia, gall bladder disease, metabolic syndrome and certain types of cancer.

Therefore in some embodiments, the nutritional composition of the present invention can be used for preventing a later in life health disorder selected from the list consisting of overweight, obesity, cardiovascular disease, hypertension or metabolic syndrome. In a preferred embodiment, it is used to prevent later in life obesity.

Another object of the present invention refers to the use of a nutritional composition according to the present invention for reducing the risk of development of overweight later in life in an infant or a young child.

Alternatively or simultaneously, the nutritional composition according to the present invention can be used to control food intake and/or to provide a healthy growth in an infant or a young child.

In some embodiments, the nutritional composition of the present invention may also promote lean mass increase in an infant or young child.

In some embodiments, the nutritional composition of the present invention may also increase the satiety responsiveness in an infant or young child.

The health benefits targeted in the present invention may be obtained with the nutritional composition by increasing GLP-1 secretion in said infant or young child, especially the intestinal GLP-1 secretion.

The health benefits targeted in the present invention may be obtained with the nutritional composition by increasing the satiety responsiveness in said infant or young child.

These represent new clinical situations where prevention of later in life health fat mass disorders like obesity can be targeted in new ways.

The invention also refers to the use of at least one sialylated oligosaccharide or a nutritional composition (as previously detailed) comprising at least one sialylated oligosaccharide for increasing GLP-1 secretion in an infant or a young child.

### Other Objects:

Another object of the present invention is the use of at least one sialylated oligosaccharide in the preparation of a nutritional composition for reducing and/or avoiding excessive fat mass accumulation in an infant or a young child and/or for preventing a later in life health disorder related to excessive fat mass accumulation in an infant or a young child, wherein said later in life health disorder is later in life obesity or a related comorbidity selected from the list consisting of cardiovascular disease, hypertension, dyslipidemia, sleep apnea, arthritis, hyperuricemia, gall bladder disease and metabolic syndrome.

A particular object of the present invention is the use of at least one sialylated oligosaccharide in the preparation of a nutritional composition for preventing overweight in an infant or a young child.

Another object of the present invention is the use of at least one sialylated oligosaccharide in the preparation of a nutritional composition for reducing and/or controlling food intake in an infant or a young child and/or for promoting a healthy growth in an infant or a young child.

Another object of the present invention is a pharmaceutical composition comprising at least one sialylated oligosaccharide for use in reducing and/or avoiding excessive fat mass accumulation in an infant or a young child and/or for use in preventing a later in life health disorder related to excessive fat mass accumulation in an infant or a young child, wherein said later in life health disorder is later in life obesity or a related comorbidity selected from the list consisting of cardiovascular disease, hypertension, dyslipidemia, sleep apnea, arthritis, hyperuricemia, gall bladder disease and metabolic syndrome.

A particular object of the present invention is a pharmaceutical composition comprising at least one sialylated oligosaccharide for use in preventing overweight in an infant or a young child.

Another object of the present invention is a pharmaceutical composition comprising at least one sialylated oligosaccharide for use in reducing and/or controlling food intake in an infant or a young child and/or for use in promoting a healthy growth in an infant or a young child.

Another object of the present invention is the use of at least one sialylated oligosaccharide (or a nutritional composition comprising thereof) for reducing and/or avoiding excessive fat mass accumulation in an infant or a young child.

Another object of the present invention is the use of at least one sialylated oligosaccharide (or a nutritional composition comprising thereof) for reducing and/or controlling food intake in an infant or a young child and/or for promoting a healthy growth in an infant or a young child.

Another object of the present invention refers to a method for reducing and/or avoiding excessive fat mass accumulation in an infant or a young child, and/or for preventing a later in life health disorder related to excessive fat mass accumulation in an infant or a young child, wherein said later in life health disorder is later in life obesity or a related comorbidity selected from the list consisting of cardiovascular disease, hypertension, dyslipidemia, sleep apnea, arthritis, hyperuricemia, gall bladder disease and metabolic syndrome, said method comprising administering to said infant or young child a nutritional composition comprising at least one sialylated oligosaccharide.

A particular object of the present invention refers to a method for preventing overweight in an infant or a young child, said method comprising administering to said infant or young child a nutritional composition comprising at least one sialylated oligosaccharide.

Another object of the present invention refers to a method for reducing and/or controlling food intake in an infant or a young child and/or for promoting a healthy growth in an infant or a young child, said method comprising administering to said infant or young child a nutritional composition comprising at least one sialylated oligosaccharide.

Another object of the present invention refers to the use of at least one sialylated oligosaccharide (or a nutritional composition comprising thereof) to promote lean mass increase in an infant or young child.

Another object of the present invention refers to the use of at least one sialylated oligosaccharide (or a nutritional composition comprising thereof) to increase the satiety responsiveness in an infant or young child.

Another object of the present invention refers to the use of at least one sialylated oligosaccharide (or a nutritional composition comprising thereof) to increase GLP-1 secretion in an infant or young child.

The different embodiments, details and examples previously described in the specification (e.g. related to the types and amounts of oligosaccharide, the nutritional composition, the administration, the targeted population . . . ) also apply to all these other objects.

### EXAMPLES

The following examples illustrate some specific embodiments of the composition for use according to the present invention. The examples are given solely for the purpose of illustration and are not to be construed as limitations of the present invention, as many variations thereof are possible without departing from the spirit of the invention.

### Example 1—Example of Composition

An example of the composition of a nutritional composition (e.g. an infant formula) according to the present invention is given in the below table 1. This composition is given by way of illustration only.

| | | |
|---|---|---|
| Nutrients | per 100 kcal | per litre |

| Energy (kcal) | | 100 | 670 |
|---|---|---|---|
| Protein (g) | | 1.83 | 12.3 |
| Fat (g) | | 5.3 | 35.7 |
| Linoleic acid (g) | | 0.79 | 5.3 |
| α-Linolenic acid (mg) | | 101 | 675 |
| Lactose (g) | | 11.2 | 74.7 |
| Minerals (g) | | 0.37 | 2.5 |
| Na (mg) | | 23 | 150 |
| K (mg) | | 89 | 590 |
| Cl (mg) | | 64 | 430 |
| Ca (mg) | | 62 | 410 |
| P (mg) | | 31 | 210 |
| Mg (mg) | | 7 | 50 |
| Mn (μg) | | 8 | 50 |
| Se (μg) | | 2 | 13 |
| Vitamin A (μg RE) | | 105 | 700 |
| Vitamin D (μg) | | 1.5 | 10 |
| Vitamin E (mg TE) | | 0.8 | 5.4 |
| Vitamin K1 (μg) | | 8 | 54 |
| Vitamin C (mg) | | 10 | 67 |
| Vitamin B1 (mg) | | 0.07 | 0.47 |
| Vitamin B2 (mg) | | 0.15 | 1.0 |
| Niacin (mg) | | 1 | 6.7 |
| Vitamin B6 (mg) | | 0.075 | 0.50 |
| Folic acid (μg) | | 9 | 60 |
| Pantothenic acid (mg) | | 0.45 | 3 |
| Vitamin B12 (μg) | | 0.3 | 2 |
| Biotin (μg) | | 2.2 | 15 |
| Choline (mg) | | 10 | 67 |
| Fe (mg) | | 1.2 | 8 |
| I (μg) | | 15 | 100 |
| Cu (mg) | | 0.06 | 0.4 |
| Zn (mg) | | 0.75 | 5 |
| Oligosaccharides | 3SL (mg) | 12 | 80 |
| (HMOs) | 6SL (mg) | 33 | 220 |

Table 1: an example of the composition of a nutritional composition (e.g. an infant formula) according to the present invention

### Example 2—Activation of NCI-H716 Cells by Sialylated Oligosaccharides

### Background

Glucagon-like peptide-1 (GLP-1) is a hormone secreted by enteroendocrine cells.

NCI-H716 is a cell line derived from human L-cells, the enteroendocrine cells that secrete GLP-1, and it is often used as a model of GLP-1 secretion. Unstimulated NCI-H716 cells secrete a basal level of GLP-1 and activation of the cells leads to dose-dependent increase in GLP-1 secretion. Activation of the NCI-H716 cells and subsequent release of GLP-1 can be triggered by nutrients such as palmitic acid, oleic acid, and meat hydrolysate, Gastrin Releasing Peptide (GRP), the cholinergic molecule Carbachol [1], bitter compounds such as denatonium [2], ginsenosides [3] and other molecules.

Activation of NCI-H716 cells by ionomycin, GRP, denatonium, or ginsenosides leads to increased intracellular calcium [1-3]. Elevation of intracellular calcium is therefore a marker of cell activation and in NCI-H716 cells it could be considered as an indicator of GLP-1 release.

### REFERENCES

[1] Reimer R A, Darimont C, Gremlich S, Nicolas-Metral V, Ruegg U T, Mace K: A human cellular model for studying the regulation of glucagon-like peptide-1 secretion. Endocrinology 2001, 142: 4522-4528.
[2] Kim K S, Egan J M, Jang H J: Denatonium induces secretion of glucagon-like peptide-1 through activation of bitter taste receptor pathways. Diabetologia 2014, 57: 2117-2125.
[3] Liu C, Zhang M, Hu M Y, Guo H F, Li J, Yu Y L et al.: Increased glucagon-like peptide-1 secretion may be involved in antidiabetic effects of ginsenosides. J Endocrinol 2013, 217: 185-196.

### Description of the Study

A calcium assay was used to determine if NCI-H716 cells respond to human milk oligosaccharides (sialylated oligosaccharides) or components/precursors thereof (lactose and sialic acid). In this assay, cultured NCI-H716 cells were loaded with a fluorescent calcium sensitive dye then stimulated with the HMOs. The response to the stimulus was measured by change in fluorescence ratio in consequence of intracellular calcium increase. The tested HMOs were 3′-sialyllactose (3SL) and 6′-sialyllactose (6SL). They were tested individually.

Buffer: negative control

GRP (Gastrin Releasing Peptide): positive control

Tested HMOs concentrations: 1 mg/mL, 5 mg/mL, 10 mg/mL and 20 mg/mL

Tested lactose and sialic acid concentrations: 5.5 mg/mL and 4.72 mg/mL respectively (correspond to the amounts contained in 10 mg/mL of 3SL, in order to make a suitable comparison with 10 mg/mL of 3SL)

### Results

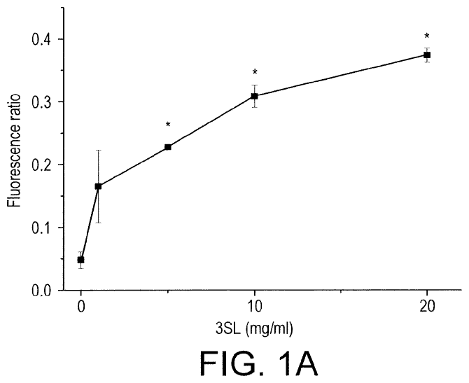

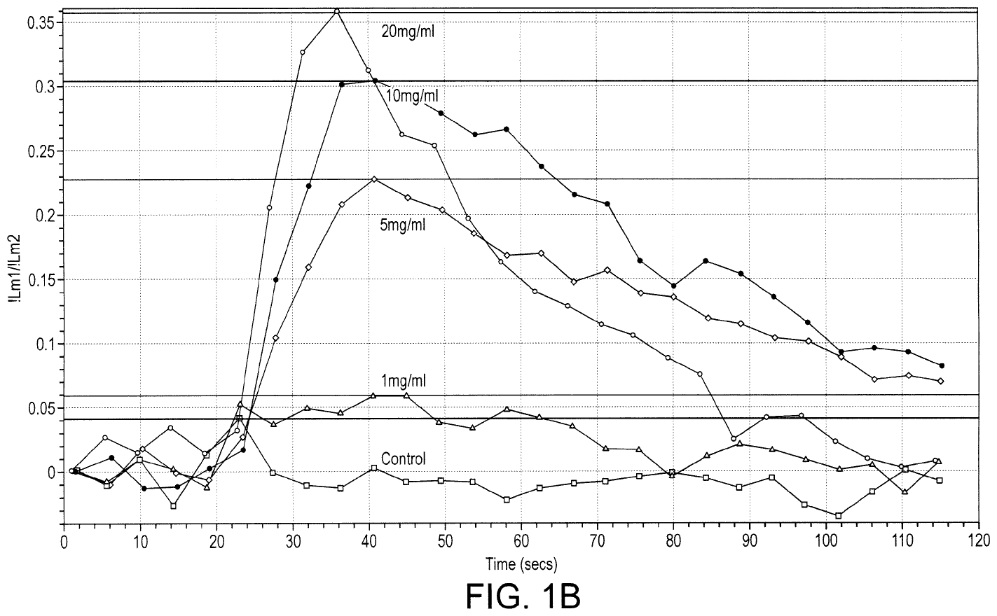

The fluorescence ratio was enhanced for 3SL and for all the tested concentrations. The increase was significant with 5 mg/mL, 10 mg/mL and 20 mg/mL (p<0.05 vs Negative control; two-sided t-test). Stimulation of NCI-H716 cells with 3SL showed a clear concentration dependent calcium response (FIG. 1).

Lactose and sialic acid, two molecular components of 3SL, did not activate the cells, showing that the response to 3SL was specific (FIG. 2). Activation of the cells with 3SL was demonstrated in four independent experiments.

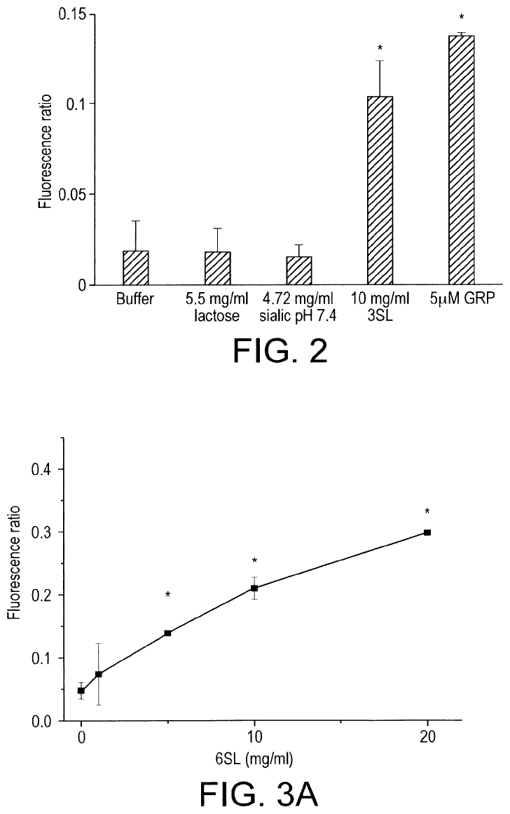

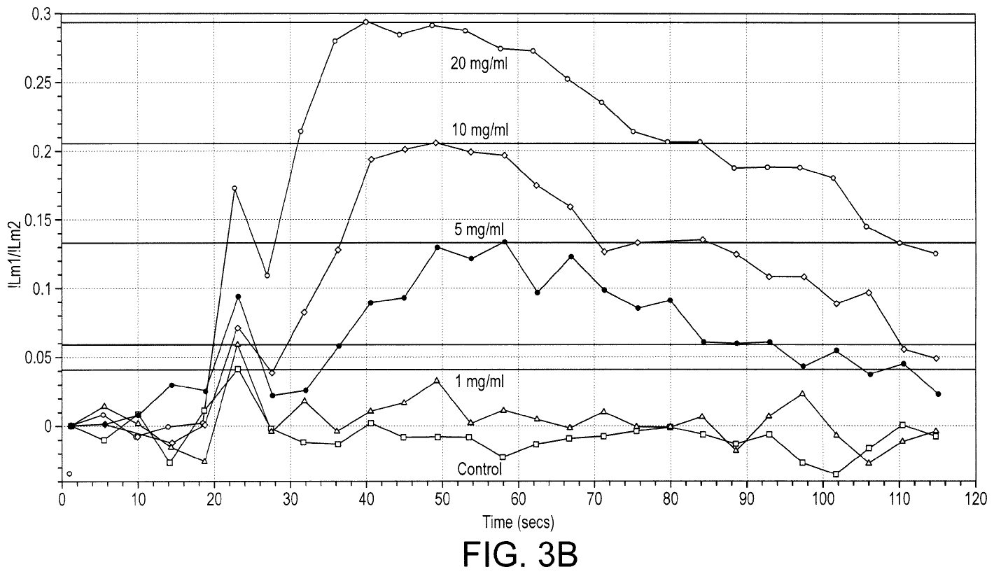

Similarly, the fluorescence ratio was enhanced for 6SL, and for all the tested concentrations. The increase was significant with 5 mg/mL, 10 mg/mL and 20 mg/mL (p<0.05 vs Negative control; two-sided t-test). Stimulation of NCI-H716 cells with 6SL also showed a clear concentration dependent calcium response (FIG. 3).

1. A method for reducing and/or avoiding excessive fat mass accumulation in an infant or a young child and/or for use in preventing a later in life health disorder related to excessive fat mass accumulation in an infant or a young child, wherein the later in life health disorder is later in life obesity or a related comorbidity selected from the group consisting of cardiovascular disease, hypertension, dyslipidemia, sleep apnea, arthritis, hyperuricemia, gall bladder disease and metabolic syndrome comprising administering a nutritional composition comprising at least one sialylated oligosaccharide to an infant or young child in need of same.

2. A method for use in reducing and/or controlling food intake in an infant or a young child and/or for use in promoting a healthy growth in an infant or a young child comprising administering a nutritional composition comprising at least one sialylated oligosaccharide to an infant or young child in need of same.

3. Method according to claim 1, wherein the sialylated oligosaccharide is selected from the group consisting of 3′-sialyllactose (3′-SL), 6′-sialyllactose (6′-SL) and a mixture thereof.

4. Method according to claim 1, wherein the sialylated oligosaccharide(s) is/are present in a total amount of from 0.1 to 10 wt %.

5. Method according to claim 1, comprising at least another oligosaccharide(s) and/or fiber(s) and/or precursor(s) of human milk oligosaccharide(s) selected from the group consisting of fucosylated oligosaccharide(s), N-acetylated oligosaccharide(s), GOS, FOS, XOS, inulin, polydextrose, sialic acid, fucose and any combination thereof.

6. Method according to claim 1, comprising at least another oligosaccharide selected from the group consisting of 2′-fucosyllactose (2FL), 3′fucosyllactose (3FL), difucosyllactose (DFL), lacto-N-tetraose (LNT), lacto-N-neotetraose (LNnT) and any combination thereof.

7. Method according to claim 1, wherein the composition further comprises at least one probiotic in an amount of from 10³ to 10¹² cfu/g of the composition (dry weight).

8. Method according to claim 1, wherein the nutritional composition is in a form selected from the group consisting of an infant formula, a starter infant formula, a follow-on or follow-up infant formula, a baby food, an infant cereal composition, a fortifier and a supplement.

9-11. (canceled)

12. Method according to claim 1, wherein the infants or young children meet at least one of the following criteria:
they have difficulties with controlling their food intake or they have an abnormal food intake such as:
i) they eat more than other infants or young children of the same age and of the same size; and/or
ii) they have an altered perception of satiety; and/or
they have an excessive weight gain during the first few months of life.

13-18. (canceled)

19. Method according to claim 2, wherein the sialylated oligosaccharide is selected from the group consisting of 3′-sialyllactose (3′-SL), 6′-sialyllactose (6′-SL) and a mixture thereof.

20. Method according to claim 2, wherein the sialylated oligosaccharide(s) is/are present in a total amount of from 0.1 to 10 wt % of the nutritional composition.

21. Method according to claim 2, comprising at least another oligosaccharide(s) and/or fiber(s) and/or precursor(s) of human milk oligosaccharide(s) selected from the group consisting of fucosylated oligosaccharide(s), N-acetylated oligosaccharide(s), GOS, FOS, XOS, inulin, polydextrose, sialic acid, fucose and any combination thereof.

22. Method according to claim 2, comprising at least another oligosaccharide selected from the group consisting of 2′-fucosyllactose (2FL), 3′fucosyllactose (3FL), difucosyllactose (DFL), lacto-N-tetraose (LNT), lacto-N-neotetraose (LNnT) and any combination thereof.

23. Method according to claim 2, wherein the composition further comprises at least one probiotic in an amount of from 10³ to 10¹² cfu/g of the composition (dry weight).

24. Method according to claim 2, wherein the nutritional composition is in a form selected from the group consisting of an infant formula, a starter infant formula, a follow-on or follow-up infant formula, a baby food, an infant cereal composition, a fortifier and a supplement.

25. Method according to claim 2, wherein the infants or young children meet at least one of the following criteria:
they have difficulties with controlling their food intake or they have an abnormal food intake such as:
i) they eat more than other infants or young children of the same age and of the same size; and/or
ii) they have an altered perception of satiety; and/or
they have an excessive weight gain during the first few months of life.
